(19) United States
(12) Patent Application Publication
(10) Pub. No.: US 2024/0201477 A1
(43) Pub. Date: Jun. 20, 2024

(54) MICROSCOPE

(71) Applicant: LEICA MICROSYSTEMS CMS GMBH, Wetzlar (DE)

(72) Inventors: Patric PELZER, Wetzlar (DE); Claus BENDER, Wetzlar (DE)

(21) Appl. No.: 18/287,315

(22) PCT Filed: Apr. 19, 2022
(86) PCT No.: PCT/EP2022/060264

§ 371 Date: Oct. 18, 2023

(30) Foreign Application Priority Data
Apr. 23, 2021 (EP) 21170309.5
Jul. 15, 2021 (EP) 21185712.3

(51) Int. Cl. G02B 21/26; G02B 21/06

(52) U.S. Cl. CPC G02B 21/26; G02B 21/06

(57) ABSTRACT

A microscope for examining a sample includes a microscope stage for receiving the sample to be examined and further includes a microscope housing enclosing the microscope stage. The microscope housing includes a first access door and a second access door, whereby both the first access door and the second access door, upon opening, provide different ways of access to the microscope stage.

### CROSS REFERENCE TO RELATED APPLICATIONS

This application is a U.S. National Phase application under 35 U.S.C. § 371 of International Application No. PCT/EP2022/060264, filed on Apr. 19, 2022, and claims benefit to European Patent Application No. EP 21170309.5, filed on Apr. 23, 2021 and European Patent Application No. EP 21185712.3, filed on Jul. 15, 2021. The International Application was published in English on Oct. 27, 2022 as WO 2022/223529 A1 under PCT Article 21(2).

### FIELD

The present invention generally relates to microscopes for examining a sample, more particularly, for imaging a sample.

### BACKGROUND

In the field of microscopic examination of living samples, like cells, it is common practice to make use of incubated sample chambers and/or to supply a reagent to the sample before or during examination of the sample. Such reagents are supplied to a sample for triggering biological or chemical reactions with the sample or just for preserving the sample. Usually, the sample itself is located in multi-well-plates, Petri dishes or microfluidic systems on a microscope table/microscope stage. Pumps may be used for supplying the reagent to the sample, on the other hand, manual pipetting may be carried out.

Additionally or alternatively, the sample is examined in an incubation atmosphere which provides best possible conditions to a living sample regarding temperature, humidity and gas, particularly CO₂, composition. While access to the sample chamber is often needed for supplying a reagent to the sample and/or for replacing the sample, and sometimes also for adjusting optical components of the microscope in the sample chamber, escape of incubation atmosphere should be limited.

Furthermore, in general, the sample handling, particularly exchanging of samples, manipulating samples, supplying reagent to the sample etc., should be facilitated.

US 2006/0092506 A1 discloses a microscope including a high humidity space that is maintained at a predetermined gas temperature for culture of a living sample. An embodiment of this microscope has three spaces, namely a cell culture space of a controlled incubation atmosphere where temperature, humidity and CO₂-concentration are controlled, an observation space including the objective lens of the microscope, the observation space being only temperature controlled, and an outside space provided with an imaging lens and a digital camera, on the one hand, and a light source for transmissive lighting, one the other hand. The cell culture space is covered with a cover box, which serves as an opening/closing unit and is provided with a thermal insulation at its inside. The cover box is provided with a hinge so that the cover box can be opened/closed with rotation around the hinge. Thus, the sample can be taken in and out from the cell culture space by opening the hinged cover box. When the cover box is opened, the cell culture space communicates with the outside space.

### SUMMARY

In an embodiment, the present disclosure provides a microscope for examining a sample, the microscope including a microscope stage for receiving the sample to be examined and further including a microscope housing enclosing the microscope stage. The microscope housing includes a first access door and a second access door, whereby both, the first access door and the second access door, upon opening, provide different ways of access to the microscope stage.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 schematically shows an embodiment of the present invention where the first access door is in its opened state;

FIG. 2 schematically shows an embodiment of the present invention where the first access door is closed;

FIG. 3 schematically shows the embodiment of FIG. 2 where the second access door is opened;

FIG. 4 schematically shows an embodiment similar to that of FIG. 2;

FIG. 5 schematically shows an embodiment where all doors of the microscope are closed;

FIG. 6 schematically shows an embodiment where the first access door is closed while the second access door is opened;

FIG. 7 schematically shows another embodiment of a microscope where all the doors are closed;

FIG. 8 schematically shows the embodiment of FIG. 7 in an opened state;

FIG. 9 schematically shows an embodiment similar to that of FIG. 7 showing a microscope with closed doors and including an incubation module adapted at its backside;

FIG. 10 schematically shows the embodiment of FIG. 9 with an opened first access door; and

FIG. 11 schematically shows the embodiment of FIG. 9 with an opened second access door.

### DETAILED DESCRIPTION

In view of the above tasks in modern microscopy, especially for examining living samples, there is a need for an improved microscope.

Embodiments of the present invention generally relate to microscopes for examining a sample, and inparticular embodiments, for imaging a sample, the microscope comprising a microscope stage for receiving the sample to be examined and further comprising a microscope housing enclosing the microscope stage. Such microscopes can particularly be used for examination of a sample in a sample chamber, which is incubated with an incubation atmosphere adapted to the sample. In general, however, also other uses and applications of such microscopes can be envisaged.

Embodiments of the present invention provide a microscope for examining a sample, the microscope comprising a microscope stage for receiving the sample to be examined and further comprising a microscope housing enclosing the microscope stage, wherein the microscope housing comprises a first access door and a second access door, whereby both, the first access door and the second access door, upon opening provide different ways of access to the microscope stage, or, in other words, different ways of direct access into the interior of the microscope housing. In principle, the first and second access doors can be arranged at the same side of the microscope housing, particularly at its front side, or at opposite sides of the microscope housing, and in a particular embodiment, at opposite sidewalls of the microscope housing, or one access door at the front side, the other one at a sidewall of the microscope housing. Further, in principle, the first and the second access doors can be of the same size or of different sizes as long as the first and the second access doors, in their opened state, provide different ways of access to the microscope stage.

The term “different ways of access to the microscope stage” includes the possible ways to reach the microscope stage through respective openings provided by the first and second opened access doors. “Different ways of access” thus require different sizes of the respective openings and/or different locations of the respective openings provided by the opened access doors. One or more different access door parameters (like at least location and size) result in different spherical angles having their base point on the microscope stage and enveloping the possible ways of access to the microscope stage through an opened access door. A user of the microscope can thus decide which access door to use according to the current demands of accessibility. These demands typically depend on whether the user needs direct access to the sample, direct access to other areas next to the microscope stage or on the microscope stage, e.g. for placing an accessory device like a pump or a reagent container next to the microscope stage, or pipetting a sample or exchanging a sample or adjusting other microscope components in the sample chamber around the microscope stage, e.g. an illumination unit or parts of an incubation system. For example, opening a sidewall door may have a lower impact on the incubation atmosphere than opening a front side door of the microscope housing. In another example, setting up an accessory device that should be placed next to the microscope stage may require access through a larger door than exchanging a sample after incubation atmosphere equilibration has been reached.

In an embodiment of the invention, the first access door is configured by a first housing part of the microscope housing and/or the second access door is configured by a second housing part of the microscope housing. Thus, an access door may be hinged housing part, but an access door may also be a slidable door in an opening of the housing. It should be noted, however, that any access door (in the meaning of the present invention), at least in its closed state, can be regarded as a housing part as it contributes to the “housing” and, in its opened state, provides direct access into the interior of the microscope housing.

In an embodiment of the invention, the first housing part is larger than the second housing part and/or the opening provided by opening the first access door is larger than the opening provided by opening the second access door. Thus, these openings can be dimensioned in a way to provide easy access to the microscope stage and its surroundings through the first access door, and, especially in case of an incubated sample chamber, to provide a more direct access to the sample itself through the second access door while minimizing disturbances of the sample inside the sample chamber, minimizing light contamination from ambient light and/or minimizing disturbances and an escape of incubation atmosphere in the sample chamber.

For ease of illustration but without loss of generality, the following embodiments of a microscope according to the present invention are based on a microscope configuration where the microscope stage is enclosed by the microscope housing to form a separate sample chamber. This sample chamber may be incubated. Other microscope components like an illumination unit may also be arranged inside the sample chamber. Such a microscope configuration may further comprise another space separated from the sample chamber and accommodating other microscope components like imaging optics. Such a configuration is typically used for transmitted-light microscopes typically used for the examination of living samples. As noted above, other configurations of a microscope according to the present invention are possible and are not meant to be excluded. For instance, the illumination unit of the microscope may be placed outside of the sample chamber and/or an incident-light microscope can be used.

Further, it is noted that the first and the second access doors according to embodiments of the present invention need not to be arranged separated from each other but rather one access door may be part of the other access door. Especially in the embodiment where the first housing part or the first access door is larger than the second housing part or the second access door, the second access door may be included in the first access door. Such a configuration may be called “door-in-door”-configuration.

It is expedient to configure the second access door as a hinged door or as a sliding door. While a sliding door can be configured to consume very little space when opened, a hinged door may be, in a preferred embodiment, configured such that the door is pivoted down in the opened state to form a horizontal or essentially horizontal work surface. Such a work surface can provide a storage area, a table or a shelf, and can thus be used for putting down things like reagent containers, laboratory bottles, pipettes, sample containers like multi-well-plates, Petri dishes and the like. Apart from its function to provide access to the microscope stage, the hinged door can thus have a second function of providing a work surface.

In another embodiment of the present invention, the microscope comprises a third door which, in its closed state, covers the second access door when in its closed state. Generally, but particularly also in this embodiment, it is advantageous if the second access door is formed in a front panel of the housing. The second and the third doors can form a “double-wall”—configuration wherein the third door needs to be opened in order to gain access to the second access door, which, in its opened state, provides access to the microscope stage. In a preferred embodiment, the second access door is, at least in part, transparent such that a user is able to visually control the status of the sample on the microscope stage without the need to open the second access door. In this context, it should be noted that it may be generally of advantage to provide at least one of the access doors with an inspection window and/or to use a transparent material enabling a user to look through the transparent access door.

In the above embodiment of a microscope comprising a third door it is particularly advantageous to form the second access door as a transparent sliding door and to form the third door as an opaque door, which is particularly larger than the second access door and which is, in a preferred embodiment, a hinged door. Having an opaque third door protects the sample from light contamination from ambient light as soon as the third door is in its closed state when having a transparent second access door.

It is further advantageous if the third door, in its closed state, is embodied as at least a part of a front cover of the microscope housing, the second access door serving for opening and closing the corresponding opening in the microscope housing. Generally, the second access door is, in a preferred embodiment, formed in a first panel of the microscope housing. In the embodiment of a microscope comprising the third door, it is preferred if the third door covers the part of the front panel including the second access door when the third door is in its closed state. In a preferred embodiment, the third door forms at least a part of a front cover such that the microscope forms a closed entity, particularly in the form of a block, when the third door is closed. The “double-wall” configuration will be further described below in connection with the figures.

In another embodiment, the microscope comprises a third door which includes the second access door. Instead of having a “double-wall” configuration, this configuration corresponds to a “door-in-door” configuration in respect of the second access door and the third door. By opening the second access door a corresponding opening is uncovered, which provides access to the microscope stage. Opening the third door uncovers a corresponding opening, which at least corresponds to the opening uncovered by the second access door. The opening uncovered by the third door may, however, be bigger than the one uncovered by the second access door. It may be expedient if the opening uncovered by the third door is included in a front panel of the housing of the microscope. By closing the third door, in a preferred embodiment, the front panel including said opening is covered by the third door. In this case, by opening the second access door, while the third door is in its closed state, a part of said opening in the front cover is uncovered. Examples of this embodiment will be described in more detail in connection with the figures below.

In an embodiment of the present invention, the first access door configured by the first housing part comprises a hinge to form a hinged lid (or hinged door or hinged cap or hinged hatch) of the microscope housing. The hinged lid of the microscope housing, in its opened state, provides direct access to the microscope stage by being hinged up or down or sideward. In this embodiment, it is particularly preferred if the hinged lid carries the second access door and, particularly, if a third door is present, also the third door.

In a further embodiment, the microscope comprises at least one gas springs, i.e., one or more gas springs. This at least one gas spring is configured to damp an opening of the first access door. In addition, or alternatively, the at least one gas spring is configured to damp a closing of the first access door. This allows an easy and smooth opening and/or closing of the first door while preventing damages. Depending on the requirements, in particular, the location of the hinge, the number of gas springs and/or their location and arrangement can appropriately be chosen.

By opening the hinged lid, a user opens a preferably bigger part of the microscope housing compared to an opening of the second access door and—if present—compared to an opening of the third door. Typically, the hinged lid is opened if a user needs access to the interior of the microscope housing, particularly to the interior of the sample chamber and its surroundings, for instance, to adjust optical components and/or the microscope stage. Also, this embodiment will be described in further detail below in connection with the figures.

As already discussed above, it is a preferred embodiment if the second access door forms a part of the first access door and/or if the first housing part of the microscope housing includes the second housing part of the microscope housing. This allows a user to reach the microscope stage in two different ways from the same side, particularly the front side, of the microscope.

In an embodiment where a third door of the microscope is present, it is a preferred embodiment if the third door is coupled to the first access door. In this case, the third door may be mounted to the first access door particularly by a hinge mechanism. One possible implementation of this embodiment is a “door-in-door” configuration where the third door is coupled to the first access door with the further option that the second access door is included in the third door. The third door would then also form an access door as it provides direct access to the microscope stage. Such an embodiment will also be described in further detail below in connection with the figures.

As also already discussed above, it is a preferred embodiment if the second access door is a hinged door which, in an open state, is configured as a work surface and, in a closed state, forms at least a part of the microscope housing.

In embodiments where the microscope comprises a third door it is a preferred embodiment if the third door is a hinged door which, in an open state, is configured as a work surface and, in a closed state, forms at least a part of the microscope housing. In such embodiments, especially if the third door covers or includes the second access door, it is preferred if the third door assumes the function of a work surface.

It should be noted that any doors of the microscope of the embodiments of the present invention can either be operated manually or electromechanically, e.g. by remote control or by control of a control unit of the microscope.

In a preferred embodiment, the microscope housing comprises a sample chamber, which sample chamber is bounded at least by the upper side of the microscope stage configured to receive the sample, and is further bounded at least by the first access door and by the second access door, when each of them is in the closed state. If a third door is present which includes the second door, the sample chamber is preferably also bounded at least by the third door in its closed state. In a preferred embodiment, the sample chamber comprises and/or is connected to an incubation module for incubating the sample chamber. These embodiments are further discussed in the embodiments according to the Figures below.

In another preferred embodiment, the sample chamber comprises an illumination unit for generating an illumination beam for illuminating the sample when received in the microscope stage. This embodiment of an transmitted-light microscope will be further discussed below.

As used herein the term “and/or” includes any and all combinations of one or more of the associated listed items and may be abbreviated as “/”.

Although some aspects have been described in the context of an apparatus, it is clear that these aspects also represent a description of a corresponding method, where a block or device corresponds to a method step or a feature of a method step. Analogously, aspects described in the context of a method step also represent a description of a corresponding block or item or feature of a corresponding apparatus.

The Figures are discussed comprehensively, with the same reference signs designating the same or at least structurally identical components.

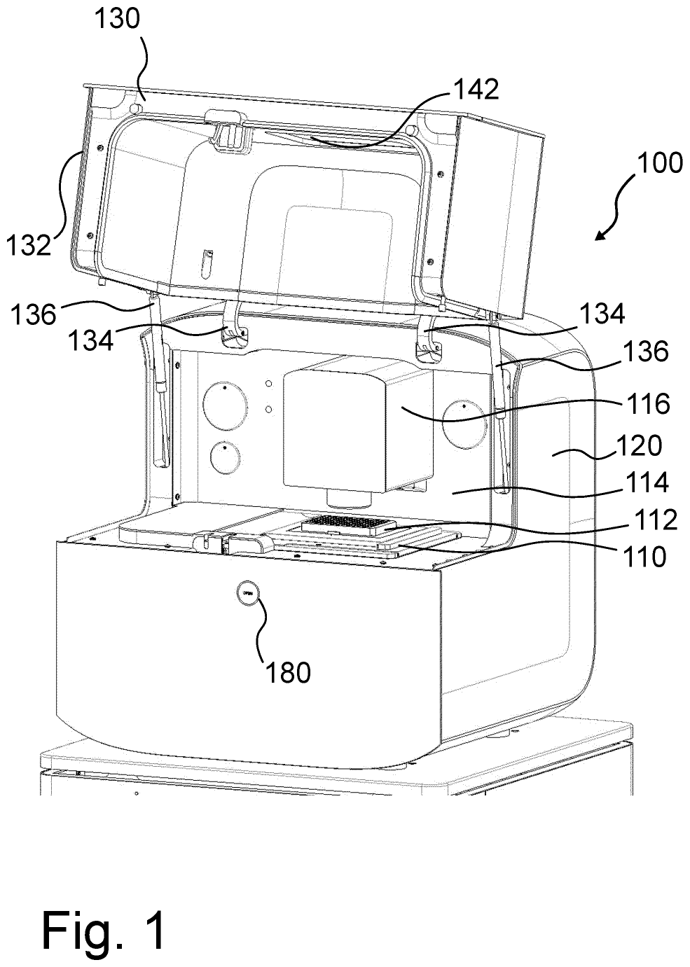

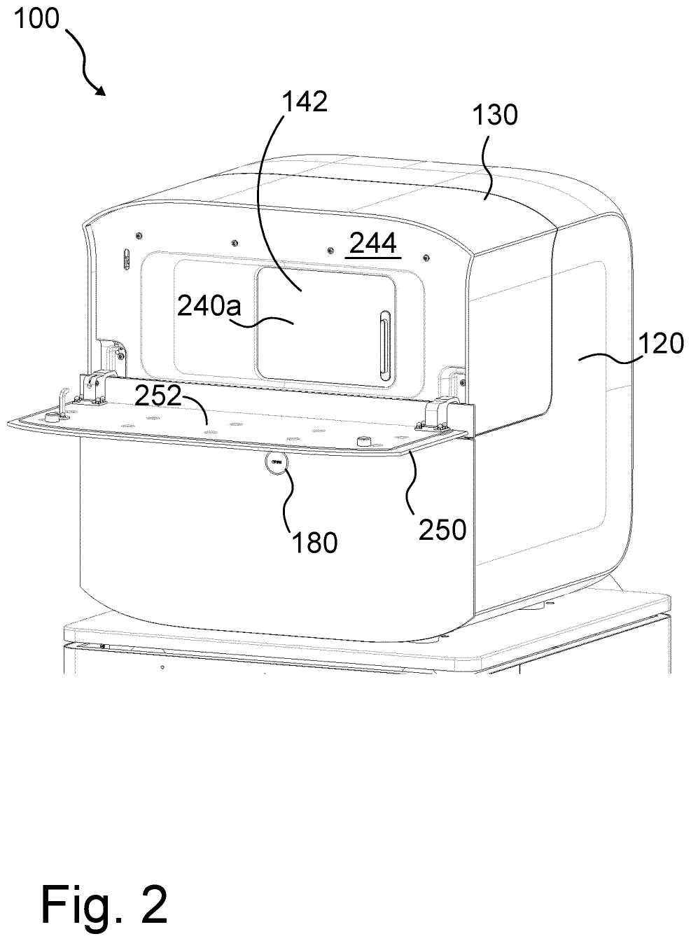

FIG. 1 schematically shows an embodiment of a microscope of the present invention, the microscope being designated 100. The microscope 100 comprises a microscope stage 110 for receiving a sample 112 to be examined/imaged and further comprises a microscope housing 120 enclosing the microscope stage 110. In this embodiment, the sample 112 is included in a multi-well-plate as shown in FIG. 1. The microscope 100 further comprises a first access door 130 configured by a first housing part 132 of the microscope housing 120. In the embodiment shown in FIG. 1, this first access door 130 is in an open state, the corresponding opening providing full access to the interior of a sample chamber 114 including the microscope stage 110. The microscope 100 further comprises a second access door generally depicted 240 (see reference sign 240a in FIG. 2) configured by a second housing part 142 of the microscope housing 120, this second housing part being formed by the closed second access door. In this embodiment, the first access door 130 includes the second access door 240a (see FIG. 2).

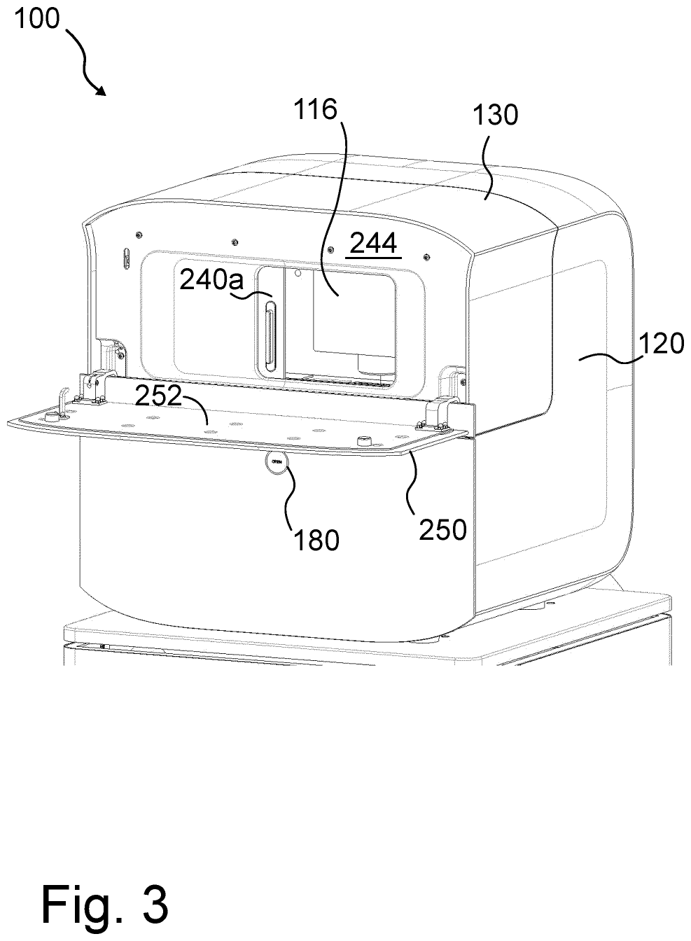

As shown in FIG. 2, the second access door 240 is a sliding door 240a, which can be opened by a sliding movement as shown in FIG. 3. As can be best seen from FIGS. 1 and 3, the first access door 130 and the second access door 240a, upon their opening, provide different ways of access to the microscope stage 110. The different sizes and the different locations of the first and second access doors result in different spherical angles enveloping possible ways of access to the microscope stage through the respective opened access doors. While the opened first access door 130 provides access to the microscope stage 110 through a wide variety of directions, the opened second access door 240a may only provide a by far restricted access to the microscope stage 110.

The embodiment shown in FIGS. 1 to 3 represent a “door-in-door” configuration as the second access door 240a is part of the first access door 130. As can be seen from FIG. 1, the first access door 130 configured by the first housing part 132 comprises hinges 134 to form a hinged lid of the microscope housing 120, opening and closing of which being damped by gas springs 136.

It is noted that, in general, a single gas spring instead of two or more gas springs can also be sufficient. In addition, it is noted that the use of such gas spring(s) does not require the specific combination of first and second access door shown in FIGS. 1 to 3, with the second access door forming part of the first access door; the effect of the gas spring(s) can also be used for the first access door only, for example, if the second access door is provided in another way or is not provided at all.

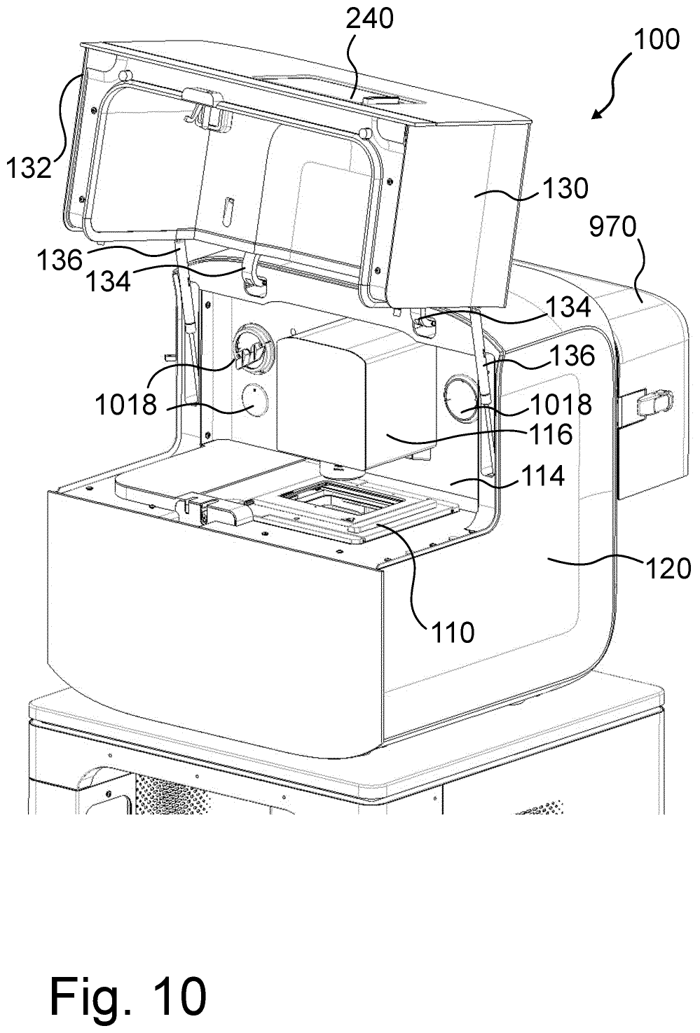

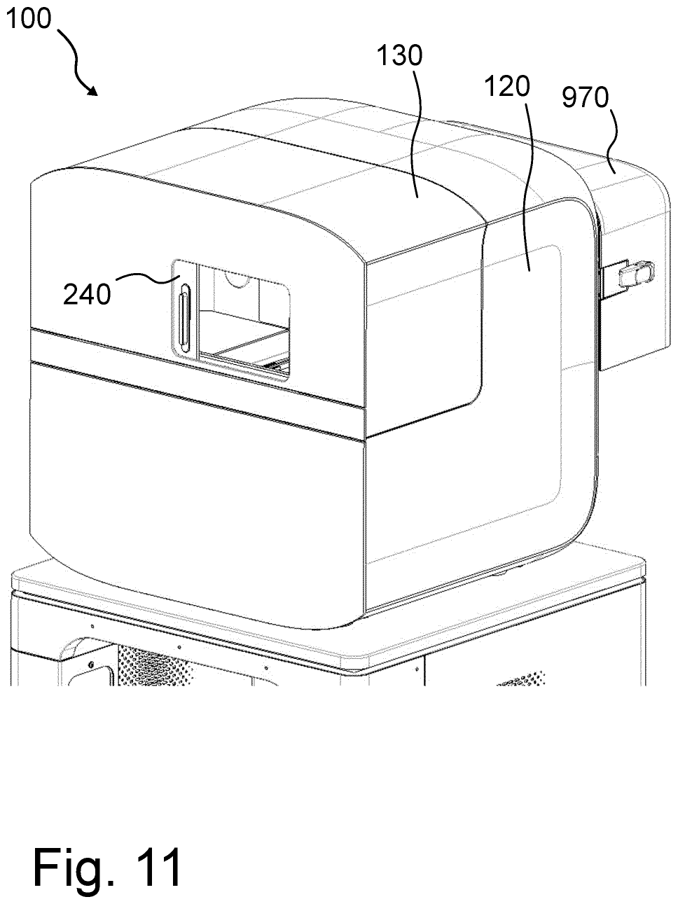

As can be seen from FIG. 1, a closed sample chamber 114 is formed by closing the first access door 130. The sample chamber 114 comprises the sample 112, the top side of the microscope stage 110 and an illumination unit 116. The closed sample chamber 114 may be incubated such that a controlled incubation atmosphere of predetermined temperature, humidity and CO₂-concentration is generated. In this embodiment of a transmitted-light microscope 100, the objective lens of the microscope 100 is arranged below the microscope stage 110. The space below the microscope stage 110 including the objective lens and possibly other imaging devices such as a digital camera may also have available a controlled atmosphere where, for example, only the temperature is controlled in order to avoid condensation of water on optical and electrical components of the imaging unit and other possible temperature-induced damages of such optical and electrical components. Regarding further details about incubation of the sample chamber 114, reference is made to the embodiments of FIGS. 10 and 11 below.

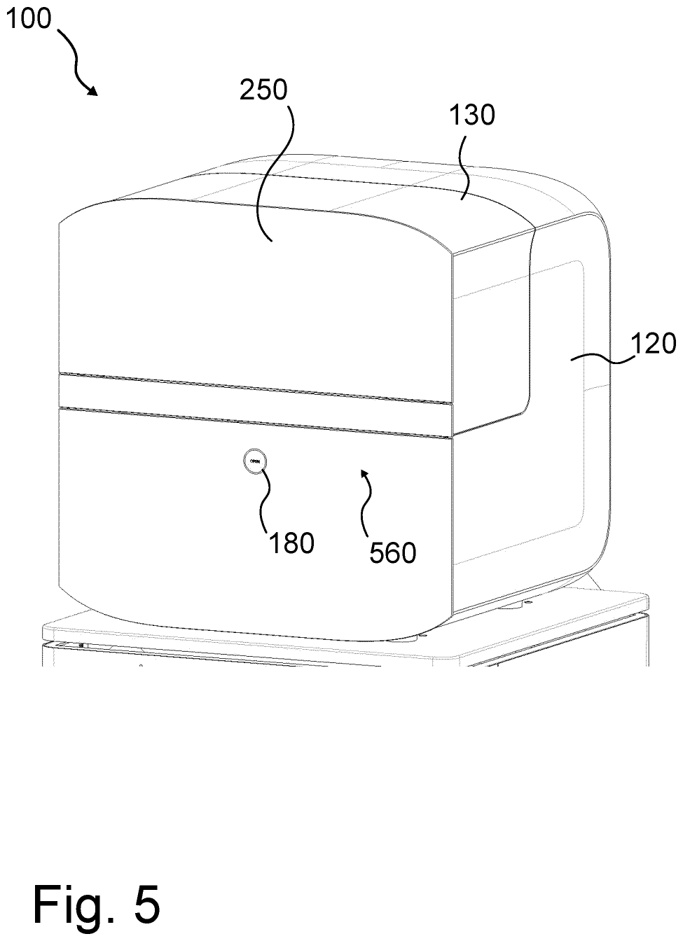

FIGS. 2 and 3 show the embodiment of FIG. 1 with a closed first access door/lid 130. The second access door 240 is a slidable door 240a. A third door 250 is provided, which, in its closed state, covers the second access door 240a when in its closed state, as can be seen from FIGS. 2 and 5. It can be seen, that the third door 250 does not provide direct access to the microscope stage 110, but the third door 250 can be used as a cover for the second access door, particularly if the second access door 240a is a transparent door, and/or can be used as a work surface 252. To this end, it is preferable if the third door 250 is a hinged door which, in an opened state, is configured as a work surface 252. This configuration can be achieved by hinging down the third door 250 which is stopped in a position as shown in FIG. 2 (opening angle of 90°). The essentially horizontal work surface 252 provides a storage area, which can be used for putting down things like reagent containers, laboratory bottles, pipettes, sample containers, etc.

If a transparent second access door 240a is used, a user can examine the interior of the sample chamber 114 while the second access door 240a is closed. Thus, no disturbance of the incubation atmosphere inside the sample chamber 114 occurs. At the same time, light contamination from ambient light is minimized. If a user wants to exchange or manipulate the sample, he/she can open the sliding door 240a. Although this results in a disturbance of the incubation atmosphere, the resulting “leakage” is much smaller compared to an opening of the first access door 130.

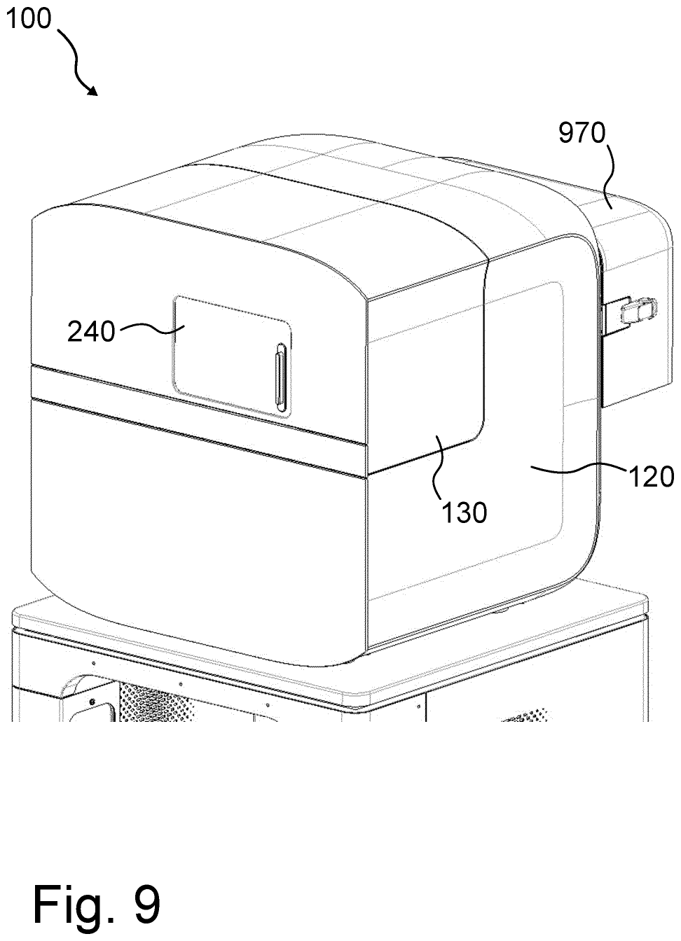

It should be noted that the second access door 240 needs not necessarily to be a sliding door but could also be another door like a hinged door. Sliding door 240a needs not to be transparent but may also be opaque in order to keep any ambient light from the sample in the sample chamber 114. In such a case, visual control of the status of the sample inside the sample chamber 114 may be done by camera inspection. In the embodiment shown in FIGS. 1 to 3, only in a limited number of cases an opening of the first access door/lid 130 is necessary. Such cases are, for example, setting up an accessory device that should be placed next to the microscope stage, adjusting the illumination unit 116 or setting up an incubation module 970 (see FIG. 9).

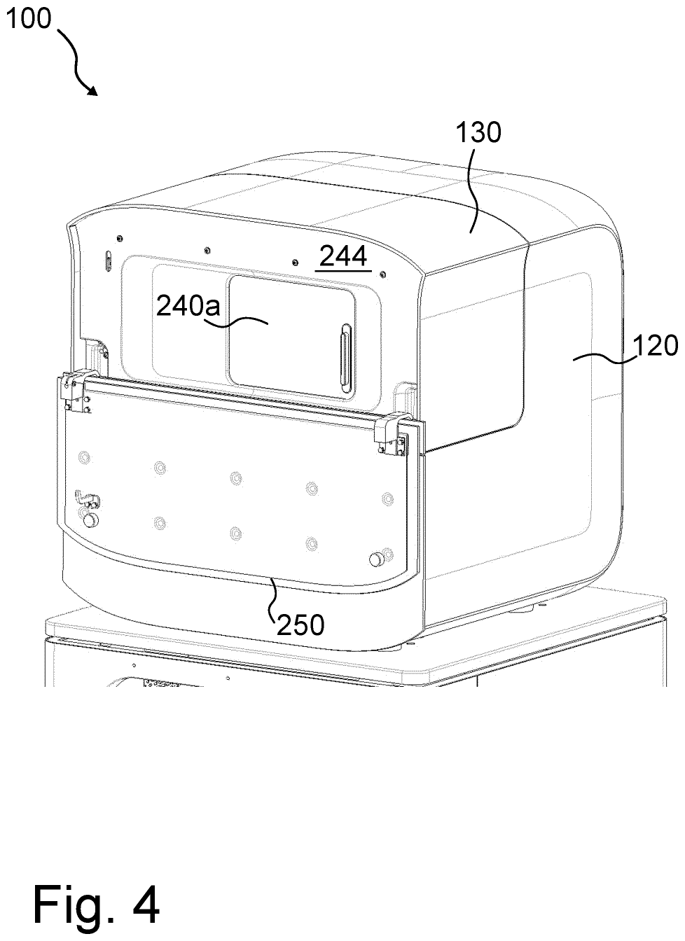

FIG. 4 shows another embodiment which is similar to that of FIG. 2 with the only difference that the second access door 250 can be hinged down by 180° instead of 90°. Although this yields no table configuration, the access benefits as discussed above are still given. This embodiment creates free space in front of the second access door 240a.

FIG. 5 schematically shows an embodiment where preferably all doors of the microscope 100 are closed. The embodiment shown in FIG. 5 can e.g. be achieved by closing the first access door 130 of the embodiment of FIG. 1 or by closing the third door 250 of the embodiments of FIG. 2, 3 or 4. As can be seen from FIG. 5, the third door 250, in its closed state, is embodied as at least a part, here the upper part, of a front cover 560 of the microscope housing 120. As can be seen from FIGS. 2 to 4, the second access door 240a may be formed in a front panel 244 of the microscope housing 120. Thus, the second access door serves for opening and closing the corresponding opening in the microscope housing. The third door 250 serves for covering the front panel 244 including the second access door 240a. In the embodiment of FIG. 5, the third door 250, in its closed state, forms the upper part of the front cover 560 which may be made of a coloured, transparent or semi-transparent glass. The microscope 100, in its closed state, forms a closed entity and has the shape of a block.

The (push) button 180 shown in FIGS. 1 to 5 serves the purpose of unlocking the first access door/lid 130 which can then be opened by a user. It is reasonable to have an interrelated locking mechanism such that the first access door 130 can only be opened if the third door 250 is closed. It may further be reasonable if the third door 250 can only be closed if the second access door 240a is closed, especially in case of an incubated sample chamber. Further, other interrelated locking mechanisms of the three doors may be provided.

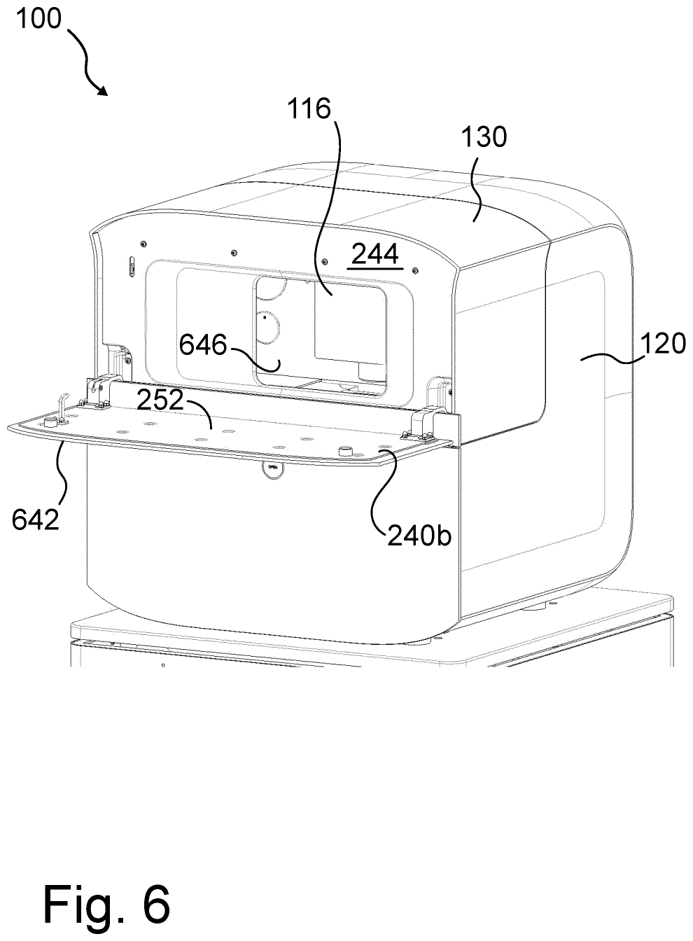

FIG. 6 schematically shows another embodiment where the first access door 130 is closed while the second access door 240 is opened. In this embodiment, the second access door 240 is a hinged door 240b. By hinging the second access door 240b down, direct access to the microscope stage 110 becomes possible. The second access door 240b is configured by a second housing part 642 serving the purpose of closing the opening 646 in the front panel 244. As the size of the opening 646 is smaller than the size of the second access door 240b, also a smaller second access door 240b may be used in this embodiment. In the embodiment shown in FIG. 6, the front panel 244 together with the closed second access door 240b form parts of the microscope housing 120. Further, in this embodiment, the second access door 240b corresponds to the third door of the embodiments of FIGS. 1 to 5, the sliding door 240a being left off. Again, a work surface 252 is formed by hinging the second access door 240b down by an opening angle of 90°. Regarding any other features and advantages, especially in regard to the first access door 130, reference is made to the previously discussed embodiments.

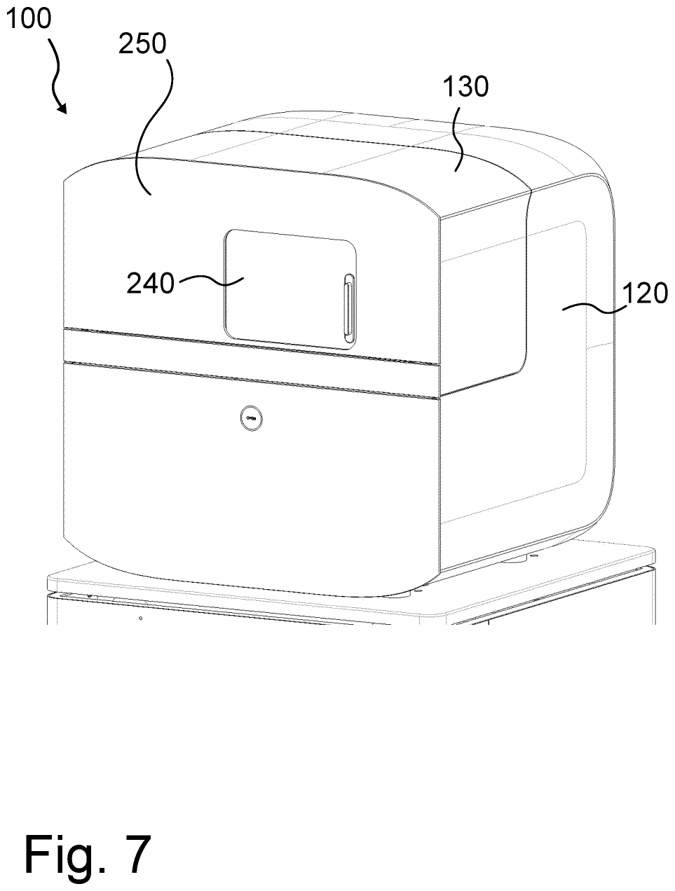

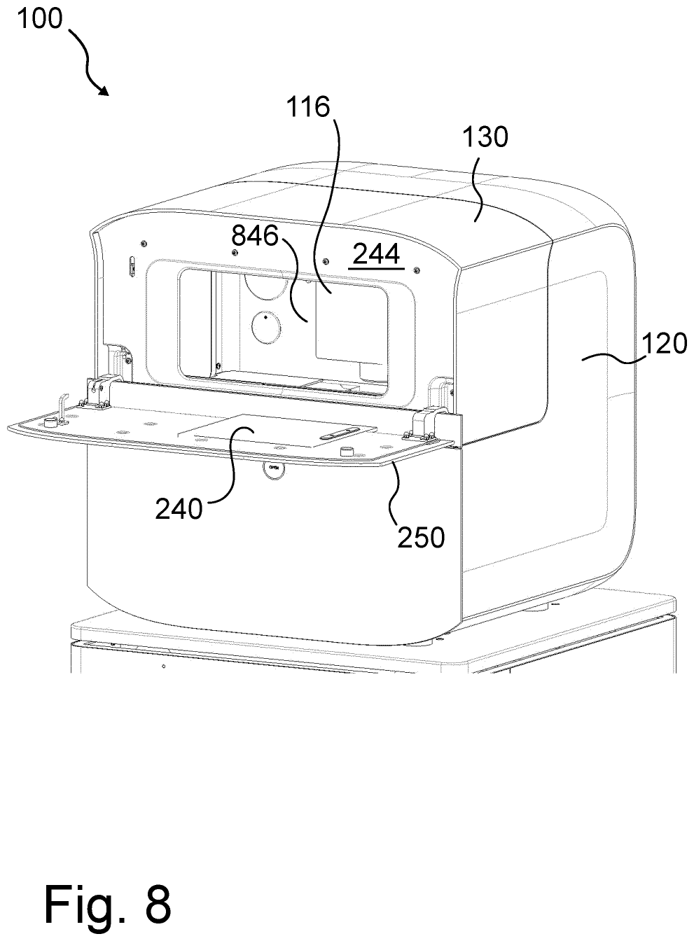

FIG. 7 schematically shows yet another embodiment of a microscope where preferably all the doors of the microscope 100 are closed. FIG. 8 schematically shows the embodiment of FIG. 7 in an opened state where the third door 250 is opened. In this embodiment, the microscope 100 comprises a third door 250 which includes the second access door 240. This configuration corresponds to a “door-in-door” configuration in respect of the second access door and the third door, which third door also has the function of an access door. By opening the second access door 240, which is configured as a sliding door, a corresponding opening is uncovered, which provides direct access to the microscope stage 110. In addition, opening the third door 250 uncovers a corresponding opening, which—as can be seen from FIG. 8—exceeds the size of the opening uncovered by the second access door 240. In this embodiment, the opening uncovered by the third door 250 is included in a front panel 244 of the microscope housing 120, however, it would also be possible to enlarge this opening by reducing or even omitting the front panel 244. On the other hand, the size of the third door 250 may be adapted to the size of the opening 846. In the embodiment shown in FIG. 8, the third door 250 corresponds to the third door shown in the embodiments of FIGS. 1 to 3, the third door 250, however, including the second access door 240 as can be seen in FIGS. 7 and 8.

In this embodiment, three different ways of access to the microscope stage 110 are provided to a user. By opening the first access door 130, a wide spherical angle of access to the microscope stage 110 is provided to a user, by opening the third door 250 uncovering the opening 846, a smaller spherical angle of access to the microscope stage 110 is provided, and by opening the second access door 240 an again smaller spherical angle of access is provided. Thus, depending on a user's intention, the user can decide among three different ways of accessibility while, at the same time, trying to keep the level of disturbance of the incubation atmosphere as low as possible.

As can be seen from FIG. 7, as the second access door 240 is part of the third door 250, and the third door 250 is part of the first access door 130, this configuration forms a “door-in-door” configuration in regard to the first, second and third doors. The third (access) door 250 is coupled to the first access door 130 by a hinge mechanism as already explained in connection with previous embodiments.

FIGS. 9 to 11 schematically show an embodiment which is similar to the embodiment of FIGS. 7 and 8 with the main difference that an incubation module 970 is adapted at the backside of the microscope 100. FIG. 9 shows this embodiment with closed doors 240 and 130, while FIG. 10 shows this embodiment with an opened first access door/lid 130, and FIG. 11 shows this embodiment with a closed first access door 130 but with an opened second access door 240. Regarding the features and advantages of the microscope 100 according to this embodiment, reference is made to the discussion of the previous embodiments in order to avoid unnecessary repetitions.

As can be seen from FIG. 10, the sample chamber 114 comprises the upper side of the microscope stage 110 and the illumination unit 116. No sample 112 has yet been placed in the microscope stage 110. As previously discussed, the microscope objective (not shown) and further components for imaging a sample, such as a digital camera, are arranged in the lower part of the microscope 100 beneath the microscope stage 110. On the backside of the sample chamber 114 three inlets/outlets 1018 are provided, two of them being closed and one of them being opened and functioning. The three inlets/outlets 1018 inter alia serve the purpose of conducting an incubation atmosphere generated by the incubation module 970 into the interior of the sample chamber 114 and/or out of the sample chamber 114. Further, sensors, tubes for pumps, etc. may be led through one or more of these inlets/outlets 1018. In the embodiment shown in FIG. 10, one of these inlets/outlets 1018 is provided with a split tube, one half of this tube being configured for conducting incubation atmosphere into the sample chamber 114, while the other part of the tube being configured for exhausting incubation atmosphere out of the sample chamber 114. In operation, a sample 112 is placed on the microscope stage 110, preferably all of the doors of the microscope 100 are closed, and the incubation module 970 is operated in order to generate an incubation atmosphere of predetermined and controlled temperature, humidity and CO₂-concentration within the sample chamber 114.

Again, depending on a user's intention, a user may only open the second access door 240 as shown in FIG. 11, for instance, in order to exchange the sample 112 or manipulate the sample 112, generating only a small “leakage” with only a small degree of air exchange between the inside and the outside of the sample chamber 114. In contrast, the user may decide to open the first access door/lid 130 for the largest possible access, for instance, for setting up the incubation module or the illumination unit. In this case, there is a high exchange of air between the inside and the outside of the sample chamber, which will most likely destroy an incubation atmosphere if already existing. On the other hand, the user might steer a middle course by opening the third door 250 of the embodiment shown in FIG. 8 if this option is implemented in the embodiment of FIGS. 9 to 11.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE SIGNS

100 microscope
110 microscope stage
112 sample
114 sample chamber
116 illumination unit
120 microscope housing
130 first access door
132 first housing part
134 hinge
136 gas spring
142 second housing part
180 button
240 second access door
240a second sliding access door
240b second hinged access door
244 front panel
250 third door
252 work surface
560 front cover
646 opening
642 second housing part
846 opening
970 incubation module
1018 inlet/outlet

1. A microscope for examining a sample, the microscope comprising:
a microscope stage for receiving the sample to be examined; and
a microscope housing enclosing the microscope stage,
wherein the microscope housing comprises a first access door and a second access door, whereby both the first access door and the second access door, upon opening, provide different ways of access to the microscope stage.

2. The microscope according to claim 1, wherein a first opening provided by opening the first access door is larger than a second opening provided by opening the second access door.

3. The microscope according to claim 1, wherein the first access door is configured by a first housing part of the microscope housing and/or the second access door is configured by a second housing part of the microscope housing.

4. The microscope according to claim 3, wherein the first housing part is larger than the second housing part.

5. The microscope according to claim 1, wherein the second access door is a hinged door or a sliding door.

6. The microscope according to claim 1, wherein the microscope further comprises a third door which, in the third door's closed state, covers the second access door when in the second access door's closed state.

7. The microscope according to claim 6, wherein the third door, in the third door's closed state, is embodied as at least a part of a front cover of the microscope housing.

8. The microscope according to claim 1, wherein the second access door is formed in a front panel of the microscope housing.

9. The microscope according to claim 1, wherein the microscope comprises a third door which comprises the second access door.

10. The microscope according to claim 1, wherein the first access door comprises a hinge to form a hinged lid of the microscope housing.

11. The microscope according to claim 10, further comprising at least one gas spring, wherein the at least one gas spring is configured to damp at least one of: opening the first access door, or closing the first access door.

12. The microscope according to claim 1, wherein the second access door forms a part of the first access door.

13. The microscope according to claim 6, wherein the third door forms a part of and/or is coupled to the first access door.

14. The microscope according to claim 5, wherein the second access door is the hinged door which, in a second access door opened state, is configured as a work surface and, in a second access door closed state, forms at least a part of the microscope housing.

15. The microscope according to claim 9, wherein the third door is a hinged door which, in a third door opened state, is configured as a work surface and, in a third door closed state, forms at least a part of the microscope housing.

16. The microscope according to claim 1, wherein the microscope housing further comprises a sample chamber, which sample chamber is bounded at least by the upper side of the microscope stage configured to receive the sample, and is further bounded at least by the first access door and by the second access door, when each of first access door and the second access door are in their respective closed state.

17. The microscope according to claim 16, wherein the sample chamber comprises and/or is connected to an incubation module for incubating the sample chamber.

18. The microscope according to claim 16, wherein the sample chamber comprises an illumination unit for generating an illumination beam for illuminating the sample when received in the microscope stage.

19. The microscope according to claim 3, wherein the first housing part of the microscope housing comprises the second housing part of the microscope housing.

20. The microscope according to claim 9, wherein the microscope housing further comprises a sample chamber, which sample chamber is bounded at least by the upper side of the microscope stage configured to receive the sample, and is further bounded at least by the first access door and by the second access door, when each of the first access door and the second access door are in their respective closed state, and
wherein the sample chamber is bounded at least by the third door in a third door closed state.
